(19) United States
(12) Patent Application Publication
(10) Pub. No.: US 2006/0270600 A1
(43) Pub. Date: Nov. 30, 2006

(54) Anti-cancer agents

(76) Inventors: Eisuke Mekada, Hirakata-shi (JP); Shingo Miyamoto, Fukuoka-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/438,767

(22) Filed: May 23, 2006

Related U.S. Application Data
(60) Provisional application No. 60/684,924, filed on May 26, 2005.

(51) Int. Cl. A61K 39/05; C07K 14/34

(52) U.S. Cl. 514/12; 424/245.1; 530/350

(57) ABSTRACT

An anti-cancer agent comprising as an active component a protein which is a diphtheria toxin mutant such as CRM197, has an activity to inhibit binding of HB-EGF to an EGR receptor and substantially does not have toxicity of diphtheria toxin. The anti-cancer agent is particularly effective for the treatment of ovarian cancer, breast cancer and prostate cancer.

### CROSS-REFERENCE TO RELATED APPLICATIONS

This application claims priority under 35 U.S.C. § 119(e) to U.S. Provisional Patent Application Ser. No. 60/684,924, filed May 26, 2005, the entire contents of which are incorporated herein by reference.

### FIELD OF THE INVENTION

The present invention relates to anti-cancer agents which can be used for treatment of entire malignant tumors such as breast cancer, prostate cancer, thyroid cancer and ovarian cancer, preferably for the treatment of the malignant tumors which express HB-EGF, and in particular can be used suitably for the treatment of ovarian cancer, breast cancer and prostate cancer.

### BACKGROUND OF THE INVENTION

Various therapeutic methods and therapeutic agents for malignant tumors have been developed, but sufficient therapeutic effects can not be achieved yet, in many cases. In particular, the ovarian cancer is the malignant tumor which makes rapid progress and particularly exhibits poor outcome in a gynecological field.

As the therapeutic method for the ovarian cancer, only a chemotherapy primarily using taxol is currently available as a measure. However, the chemotherapy exhibits tentative effects, but often allows relapsing later on, and it has been required to develop new therapeutic methods.

HB-EGF is a member of EGF family of growth factors, and has been known as a molecule which is essential for body formation and regeneration process as well as which is involved in occurrence of vascular stenosis and arteriosclerosis (e.g., see Non-patent Document 1), but this factor has not been targeted at all to date for the treatment of malignant tumors. This molecule is synthesized as a membrane bound precursor (proHB-EGF), which is then cleaved by protease on the cell surface to produce the soluble form of HB-EGF. The soluble form has a growth facilitating action whereas a growth inhibitory action is observed in the membrane type. Thus, the soluble form type and the membrane type appear to work on the formation and maintenance of tissues by being optionally used separately.

HB-EGF binds the EGF receptor (Her1) and Her4 (ErbB-4) of Erb B receptors to activate. However, members (Her1, Her2, Her3, Her4) of the Erb B family receptors form homodimers, additionally can form heterodimers in all combinations, and thus accordingly HB-EGF can activate all of the molecules of the Erb B family receptors. HB-EGF is expressed in various tissues, and appears to work in broad cells and tissues. It has been reported that HB-EGF facilitates the growth of fibroblasts, smooth muscle cells or keratinocytes (e.g., see Non-patent Document 2).

HB-EGF is synthesized as the membrane-bound precursor (proHB-EGF) as the above, and proHB-EGF is composed of a signal sequence, a prosequence, a heparin-binding domain, and EGF-like domain, a juxtamembrane domain, a transmembrane domain and a cytoplasmic domain from the N-terminus (FIG. 1). ProHB-EGF is cleaved (ectodomain shedding) by the protease at a position shown by an arrow in the figure to become the soluble form type. It has been proposed that the ectodomain shedding of proHB-EGF is stimulated by lysophosphatidic acid (LPA) through the activation of G protein-coupled receptor followed by Ras-Raf-MEK pathway or phorbol ester through the activation of PKC(e.g., see Non-patent Document 3).

The action of the soluble form of HB-EGF to bind to the EGF receptor and to induce the phosphorylation of the EGF receptor is present in the EGF-like domain (e.g., see Non-patent Document 1).

Meanwhile, it has been known that a diphtheria toxin is a protein with a molecular weight of about 59,000 produced by diphtheria bacillus, and utilizes the membrane-bound precursor of HB-EGF (proHB-EGF) as the receptor (e.g., see Non-patent Document 4). A diphtheria toxin mutant such as CRM197 has been known as an inhibitor of the soluble form of HB-EGF (e.g., see Non-patent Document 5). For database information of diphtheria toxin, a gene, an amino acid sequence and a three dimensional structure are available in EMBL;K01722, SWISS-PROT;P00588 and PDB;1MDT or 1XDT, respectively. The gene of diphtheria toxin is encoded by a phage lysogenized into diphtheria bacilli.

Diphtheria toxin is a single polypeptide protein composed of 535 amino acid residues (the amino acid sequence [SEQ ID NO:1] of diphtheria toxin and a base sequence [SEQ ID NO:2] of the gene encoding it are shown in FIGS. 2R>2 and 3, italic letters represent the signal sequence), and can be divided into a fragment A moiety and a fragment B moiety separated by treating with a reducing agent (FIG. 4), but the fragment B moiety is further divided into two domains according to conformational structure analysis. For the function of each domain, a catalytic domain corresponding to the fragment A moiety (amino acid numbers of 1 to 193 excluding the signal sequence) has an ADP ribosylation activity. A transmembrane domain corresponding to an N-terminal half of the fragment B moiety (amino acid numbers of 194 to 378 excluding the signal sequence) has a nature to form a channel in an endosome membrane. A receptor binding domain corresponding to a C-terminal half of the fragment B moiety (amino acid numbers of 386 to 535 excluding the signal sequence) has an activity to bind to the diphtheria toxin receptor on the cell surface.

The fragment A (catalytic action domain) of diphtheria toxin has an activity to ADP-ribosylate EF-2 (peptide elongation factor 2) in the presence of NAD, thereby inhibiting protein synthesis. Therefore, in order to exert toxicity of diphtheria toxin, the fragment A must enter in the cytoplasm.

For the mechanism in which the fragment A enters in the cytoplasm, diphtheria toxin is incorporated into the endosome by endocytosis by binding the receptor binding domain in the fragment B to proHB-EGF which is the receptor on the cell surface, the transmembrane domain is inserted into the endosome membrane in the endosome, and finally the fragment A passes through the endosome membrane to liberate in the cytoplasm and inactivates EF-2 there (e.g., see Non-patent Document 6).

To exert the toxicity of diphtheria toxin, both the fragment A and the fragment B are necessary. Therefore, even if either fragment is mutated, the protein having no toxicity of diphtheria toxin is made.

Non-toxic mutants of diphtheria toxin e.g., CRM197 by mutating the catalytic domain have been isolated.

Meanwhile, it is due to the binding of diphtheria toxin to the EGF-like domain of the soluble form of HB-EGF that the mutant of diphtheria toxin has the activity to inhibit the binding of HB-EGF to the EGF receptor. The receptor binding domain of diphtheria toxin is involved in this binding. It has been reported that Lys at position 516 and Phe at position 530 in diphtheria toxin are important for the binding to HB-EGF (e.g., see Non-patent Document 7). A crystal structure of a complex composed of diphtheria toxin and the EGF-like domain of HB-EGF has been analyzed, and the amino acids between positions 381 and 535 have been reported to be important for the binding to HB-EGF (e.g., see Non-patent Document 8).

This way, it has been observed that the diphtheria toxin mutant binds to HB-EGF and inhibits the activity of HB-EGF, but the use of the diphtheria toxin mutant for cancer therapeutic drugs has not been attempted at all because it has not been known that HB-EGF is targeted for cancer therapy and the control mechanism including HB-EGF is complicated.

Non-patnet Document 1: Eisuke Mekada et al., “Idenshi Igaku” Vol. 5, No. 2, P131-134, 2001, published by Medical Do Co., Ltd.

Non-patent Document 2: Higashiyama, S. et al., J. Cell Biol. (1993)122, p.933-940.

Non-patent Document 3: Prenzel, N. et al., Nature (1999) 402, p.884-888.

Non-patent Document 4: J. G. Naglich et al., Cell (1992) 69, p.1051-1061.

Non-patent Document 5: T. Mitamura et al., J. Biol. Chem. (1995)270, p.1015.

Non-patent Document 6: T. Umata et al., J. Biol. Chem. (1998)273, p.8351.

Non-patent Document 7: Shen, H S et al. J. Biol. Chem. (1994)269, p.29077-29084.

Non-patent Document 8: Gordon V L et al., Molecular Cell (1997) 1,p.67-78.

### SUMMARY OF THE INVENTION

One embodiment of the present invention is an anti-cancer agent comprising as an active component a mutant diphtheria toxin protein which inhibits binding of HB-EGF to an EGF receptor and substantially does not have toxicity of diphtheria toxin. In another embodiment, the protein comprises at least the receptor binding domain in the amino acid sequence of diphtheria toxin without having a mutation. In yet another embodiment, the protein comprises an amino acid sequence having one or more amino acid deletions, substitutions or additions in the amino acid sequence of diphtheria toxin. The protein may be either CRM197 or DT52E148K. In yet another embodiment, the anti-cancer agent neutralizes toxicity of diphtheria toxin which remians in the diphtheria toxin mutant and further comprises a monoclonal antibody against the diphtheria toxin mutant, which does not inhibit biding the diphtheria toxin mutant to cells, in which the monoclonal antibody against the diphtheria toxin mutant and the diphtheria toxin mutant form a complex. In one aspect of this embodiment, the monoclonal antibody against the diphtheria toxin mutant is #2 anti-DT mAb. In one embodiment, the cancer is ovarian cancer, breast cancer or prostate cancer. In another embodiment, the cancer is peritoneal metastitic cancer.

The present invention also provides an anti-cancer agent comprising as an active component a protein which is any of the following proteins (a), (b) or (c), inhibits binding of HB-EGF to an EGF receptor and substantially does not have toxicity of diphtheria toxin: (a) a protein comprising part of diphtheria toxin and at least the receptor binding domain of diphtheria toxin; a protein comprising an amino acid sequence having one or more amino acid deletions, substitutions or additions in the amino acid sequence of the protein (a); and (c) a complex protein comprising any of (a) and (b). In one embodiment, any of the proteins (a), (b) or (c) does not have the catalytic domain of diphtheria toxin. In another embodiment, the protein (c) is GST-DT.

### BRIEF DESCRIPTION OF THE DRAWINGS

FIG. 1 is a schematic view showing a structure of proHB-EGF.

FIG. 2 is a view showing the amino acid sequence and the base sequence of diphtheria toxin.

FIG. 3 is a view showing the amino acid sequence and the base sequence of diphtheria toxin (continued from FIG. 2).

FIG. 4 is a schematic view showing a domain structure of diphtheria toxin.

FIG. 5 is a view showing the amino acid sequence and the base sequence of GST-DT.

FIG. 6 is a view showing the amino acid sequence and the base sequence of GST-DT (continued from FIG. 5).

FIG. 7 is a view showing effects of intraperitoneal injection of CRM197 on tumor growth in nude mice in which SKOV-3 cells were injected.

FIG. 8 is a view showing effects of topical injection of CRM197 on tumor growth in nude mice in which SKOV-3 cells were injected.

FIG. 9 is a view showing effects of topical injection of CRM197 on tumor growth in nude mice in which SKOV-3 cells were injected.

FIG. 10 is a view showing effects after CRM197 injection on cancer growth rates in nude mice in which RMG-1 cells were injected.

FIG. 11 is a view showing effects after CRM197 injection on cancer growth rates in nude mice in which OV47 cells were injected.

FIG. 12 is a view showing effects after CRM197 injection on cancer growth rates in nude mice in which SKOV-H cells were injected.

FIG. 13 is a view showing toxicity for Vero cells and Vero-H cells when CRM197 was allowed to act for 24 hours.

FIG. 14 is a view showing toxicity for Vero cells and Vero-H cells when CRM197 was allowed to act for one week.

FIG. 15 is a view showing an inhibitory effect of CRM197 on protein synthesis.

FIG. 16 is a view showing toxicity of CRM197, DT52E148K and GST-DT on Vero-H cells.

FIG. 17 is a view showing toxicity of CRM197, DT52E148K and GST-DT on Vero cells.

FIG. 18 is a view showing DT resistance of Vdtr-4H cells.

FIG. 19 is a view showing ADP-ribosylating reaction of EF-2 isolated from Vdtr-4H cell and Vero-H cell lysates in a cell free condition.

FIG. 20 is a view showing resistance of Vdtr-4H cells to CRM197 and DT52E148K.

FIG. 21 is a view showing ADP-ribosylation of EF-2 by CRM197 and DT.

FIG. 22 is a view showing ADP-ribosylation of EF-2 by DT52E148K.

FIG. 23 is a view showing neutralization of toxicity of CRM197 by #2 anti-DT mAb.

FIG. 24 is a view showing inhibition of HB-EGF cell growth activity by CRM197/·2 anti-DT mAb complex.

FIG. 25 is a view showing growth of cancer cells by intraperitoneal administration of CRN197 and CRM197/#2 anti-DT mAb complex in nude mice.

FIG. 26 is a view showing growth of cancer cells by intraperitoneal administration of CRM197 and DT52E148K in nude mice.

FIG. 27 is a view showing growth of cancer cells by taxol administration in xenografted mice.

FIG. 28 is a view showing expression levels of EGFR ligand in human cancer cells.

FIG. 29 is a view showing effects after CRM197 injection on cancer growth rates in nude mice in which MDA-MB231 (breast cancer) cells were injected.

FIG. 29 is a view showing effects after CRM197 injection on cancer growth rate in nude mice in which PC-3 (prostate cancer) cells were injected.

FIG. 31 shows effects of CRM197 in a model of ovarian cancer peritoneal dissemination.

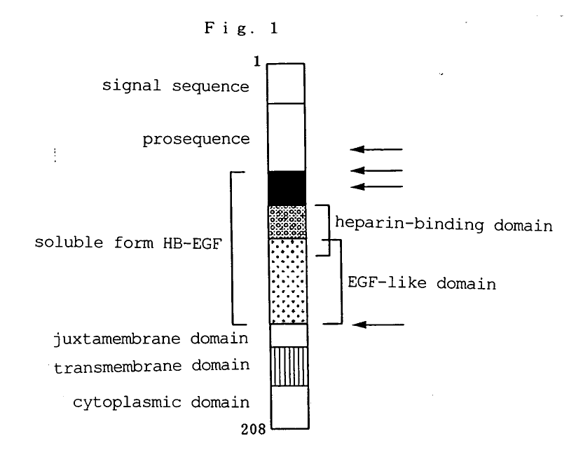

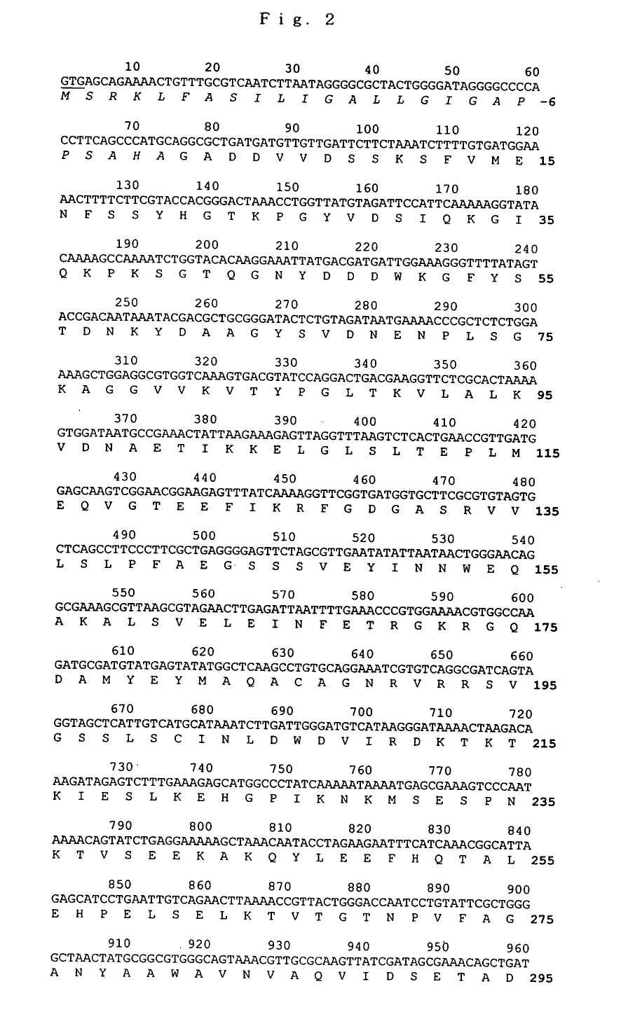

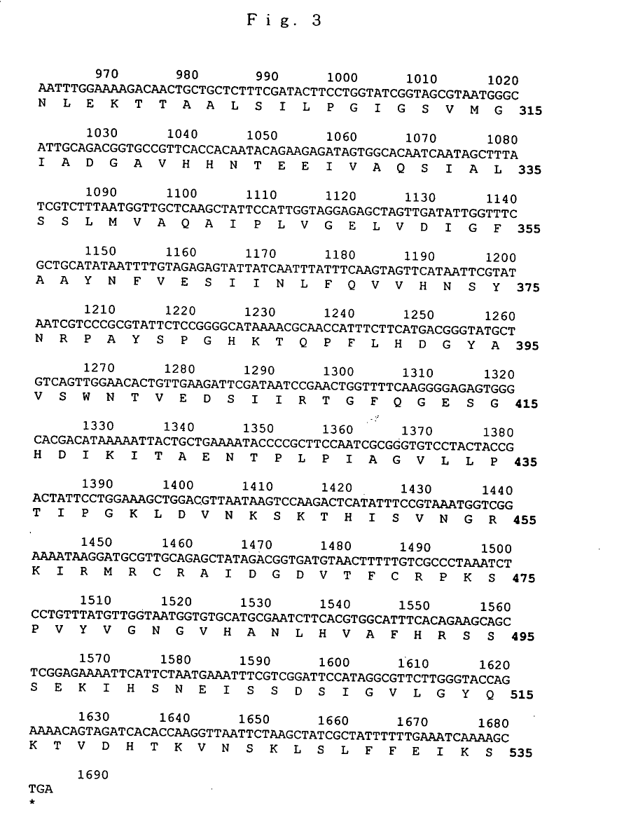

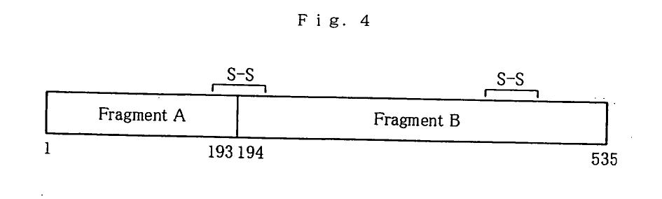

### DETAILED DESCRIPTION OF THE INVENTION

The present invention makes it a subject to solve the conventional various problems and achieve the following object. That is, it is the object of the present invention to provide anti-cancer agents effective for treatment of entire malignant tumors such as breast cancer, prostate cancer, thyroid cancer and ovarian cancer, preferably the treatment of the malignant tumors which express HB-EGF, and in particular the treatment of ovarian cancer, breast cancer and prostate cancer.

The present inventors have restudied functions of the soluble form of HB-EGF known as a protein which is essential for body formation and regeneration process as well as which is involved in occurrence of vascular stenosis and arteriosclerosis, and have obtained findings that HB-EGF is involved in growth and metastasis process of cancer cells. Based on these findings, the present inventors have carried forward development of new therapeutic methods, as a result, have demonstrated that CRM197 having a neutralizing ability of HB-EGF significantly inhibits the growth of cells derived from malignant tumors, particularly cells derived from ovarian cancer inoculated into nude mice, and have led to the present invention.

That is, means of the present invention to solve the above subject are as follows.

<1> A anti-cancer agent characterized by comprising as an active component a protein which is a diphtheria toxin mutant, has an activity to inhibit binding of HB-EGF to an EGF receptor and substantially does not have toxicity of diphtheria toxin.

<2> The anti-cancer agent according to <1> characterized in that the protein comprises at least the receptor binding domain in the amino acid sequence of diphtheria toxin without having a mutation.

<3> The anti-cancer agent according to <1> or <2> wherein the protein is composed of an amino acid sequence having one or more amino acid deletions, substiutions or additions in the amino acid sequence of diphtheria toxin.

<4> The anti-cancer agent according to <1> to <3> wherein the protein is either CRM197 or DT52E148K.

<5> The anti-cancer agent according to any of <1> to <4> having an activity to neutralize toxicity of diphtheria toxin which remains in said diphtheria toxin mutant and further comprising a monoclonal antibody against said diphtheria toxin mutant, which does not inhibit binding said diphtheria toxin mutant to cells, wherein the monoclonal antibody against said diphtheria toxin mutant and the diphtheria toxin mutant form a complex.

<6> The anti-cancer agent according to <5> wherein the monoclonal antibody against said diphtheria toxin utant is #2 anti-DT mAb.

<7> The anti-cancer agent according to any of <1> to <6> wherein a cancer subjected to the treatment is any of ovarian cancer, breast cancer and prostate cancer.

<8> The anti-cancer agent according to any of <1> to <6> wherein a cancer subjected to the treatment is peritoneal metastatic cancer.

<9> A anti-cancer agent characterized by comprising as an active component a protein which is any of the following proteins (a), (b) and (c), has an activity to inhibit binding of HB-EGF to an EGF receptor and substantially does not have toxicity of diphtheria toxin:
(a) a protein composed of a part of diphtheria toxin and comprising at least the receptor binding domain of diphtheria toxin;
(b) a protein composed of an amino acid sequence having one or more amino acid deletions, substitutions or additions in the amino acid sequence of the protein (a); and
(c) a complex protein comprising any of (a) and (b).

<10> The anti-cancer agent according to <9> wherein any of the proteins (a), (b) and (c) does not have the catalytic domain of diphtheria toxin.

<11> The anti-cancer agent according to <9> wherein the protein (c) is GST-DT.

According to the present invention, it is possible to provide the anti-cancer agent effective for the treatment of the malignant tumors (including peritoneal dissemination), particularly ovarian cancer, breast cancer and prostate cancer.

### DETAILED DESCRIPTION OF THE INVENTION

The present inventors have demonstrated the anti-cancer action of detoxified mutants of diphtheria toxin whose inhibitory activity on binding of HB-EGF to EGF receptor has been known, based on obtained findings that HB-EGF has tumorigenicity in investigation of HB-EGF functions.

The anti-cancer agent in a first embodiment of the present invention is one of the mutant forms of diphtheria toxin, and is characterized by comprising as an active component a protein having an activity to inhibit the binding of HB-EGF to the EGF receptor and substantially not having toxicity of diphtheria toxin.

The above mutant of diphtheria toxin represents the protein composed of an amino acid sequence having one or more amino acid deletions, substitutions or additions in the amino acid sequence of diphtheria toxin, and for example, is the protein composed of the amino acid sequence having one or several amino acid deletions, substitutions or additions. The signal sequence composed of 25 amino acids of diphtheria toxin may be included or need not be included, and any of the sequences is included in the scope of the present invention.

The anti-cancer agent in a second embodiment of the present invention is any of the following proteins (a), (b) and (c), and is characterized by comprising as an active component a protein having an activity to inhibit the binding of HB-EGF to the EGF receptor and substantially not having toxicity of diphtheria toxin.
(a) a protein composed of a part of diphtheria toxin and comprising at least the receptor binding domain of diphtheria toxin;
(b) a protein composed of an amino acid sequence having one or more amino acid deletions, substitutions or additions in an amino acid sequence of the protein (a); and
(c) a complex protein comprising any of (a) and (b).

The above protein refers to one which is the part of diphtheria toxin or the mutant thereof or the complex protein comprising those proteins and retains the receptor binding domain.

Here, the toxicity of diphtheria toxin means that diphtheria toxin binds to the receptor on the cell surface, enters in cells and inhibits protein synthesis in the cells by an activity to ADP-ribosylate (ADP ribosylation activity) EF2 (peptide elongation factor), which a fragment A retains, and this can be easily measured by levels of protein synthesis inhibition. That is, a certain amount of diphtheria toxin is added to cultured cells, which are then cultured for about 2 to 8 hours, subsequently cultured in the presence of a radiolabeled amino acid for a short time, and then the amount of the radiolabeled amino acid incorporated into proteins is measured.

Specifically, Vero cells (1×10⁵ cells) are seeded in a 24-well plate, and cultured in a CO₂ incubator for 16 hours. After confirming that the cells adhere thoroughly to the plate, each well is washed once with cold PBS (150 mM NaCl, 27 mM KCl, 10 mM phosphate buffer pH 7.2). The cells are washed carefully not to detach the cells from the plate. A medium (0.5 ml) for assay including serum is added. In the medium for assay, a concentration of leucine is reduced to about 1/10 from the usual medium. This is for enhancing an efficiency of uptake of [³H]-leucine added later. But, in Ham's F12 medium, the leucine content is low, and thus this can also be used as the medium for assay. The serum is added at a concentration usually used.

Subsequently, diphtheria toxin at various concentrations is added, and the cells are cultured in the CO₂ incubator for 2 to 5 hours. [³H]-leucine (10 μl) at 3.7 MBq/ml is added, and the culture is continued for an additional one hour.

The medium is discarded, wells are washed once with PBS, the cells are lysed with 0.5 ml of 0.1 M NaOH, and the lysate is collected in a tube. The well is washed again with 0.5 ml of 0.1 M NaOH, and the solution is collected in the same tube.

A solution (0.5 ml) of 20% trichloroacetic acid is added thereto, and the tube is thoroughly stirred by a Vortex mixer. A produced precipitate is collected with a grass filter and the filter is further washed with 5% trichloroacetic acid.

Finally, the filter is washed with 100% ethanol, and dried.

The filter is immersed in a scintillator of toluene/PPO, and radioactivity trapped on the filter is measured using a liquid scintillation counter. A value of a sample in which diphtheria toxin was not added is measured to make 100%, and a value when diphtheria toxin was added is calculated as %.

The protein substantially not having the toxicity of diphtheria toxin refers to the protein in which the toxicity of diphtheria toxin is completely lost or attenuated to a extremely low level, and in the present invention, refers to one having no significant difference from the value of the sample in which diphtheria toxin was not added or the sample in which diphtheria toxin mutant having no catalytic action domain was added when the toxicity of diphtheria toxin at 1 ng/ml is measured in the above Vero cell system. The above significant difference is preferably no significant difference at a significant level of 5%, more preferably no significant difference at a significant level of 1% and still more preferably no significant difference at a significant level of 0.1% in t-test.

But, in the present invention, in the mutants such as CRM197 and DT52E148K described to have no toxicity of diphtheria toxin, it has been proved that the extremely faint toxicity (e.g., in CRM197, about from 1/10⁷ to 1/10¹⁰ of wild type diphtheria toxin) remains, and the mutants having such a faint toxicity are not excluded from the present invention. It is preferable that the toxic levels of diphtheria toxin is equivalent to or lower than the level of CRM197, in terms of eliminating a side effect due to the toxicity of diphtheria toxin and enhancing the safety.

However, on the other hand, the present invention suggests that the toxicity contributes to the effect of the anti-cancer agent. Thus, it is preferable to have the toxicity at extremely low level equivalent to the level in CRM197 in terms of enhancing the anti-cancer effect. Therefore, it is possible to optionally select the toxic level of diphtheria toxin depending on drug formulation.

The toxicity of diphtheria toxin can be controlled by mutating the catalytic domain essential for ADP-ribosylation of EF-2 or deleting a part or of the entir catalytic domain.

The function of the mutated catalytic domain can be accurately examined by directly measureing the ADP-ribosylation activity. The ADP-ribosylation activity (mutated catalytic domain) and radiolabeled NAD to isolated and purified EF-2 in vitro and measuring the radioactivity incorporated in EF-2.

Specifically, at final concentrations, 20 mM tris buffer (pH 7.8), 1 mM DTT (dithiothreitol), 0.1 to 1 μg/ml of the fragment A or 0.1 to 1.00 μg/ml of the protein to measure the ADP ribosylation activity are added to an EF-2 fraction of rabbit reticulocytes obtained by the method described in the following reference (Moynihan, M. R. and Pappenheimer, A. M. Jr. Infect. Immun. (1981) 32, 575-582), further [³²P]NAD(about 740 GBq/mmol) is added at a final concentration of 370 KBq/ml, mixed and incubated at 37° C. for 10 minutes.

The same volume of 10% trichloroacetic acid solution is added to the reaction solution to precipitate the protein, a produced precipitate is collected with a glass filter, and the filter is further washed with 5% trichloroacetic acid. Finally, the filter is washed with 100% ethanol, and dried.

The filter is immersed in the scintillator of toluene/PPO, and radioactivity trapped on the filter is measured using a liquid scintillation counter.

The measured radioactivity represents the level of the ADP-ribosylation activity. Based on the radioactivity using the unmutated fragment A, it is possible to calculate a relative activity of the ADP-ribosylation activity of the mutated protein.

The more detailed study by the present inventors based on domain information have shown that the characteristic of having the activity to inhibit the binding of the soluble form of HB-EGF to the EGF receptor is obtained when the amino acid sequence from position 378 to position 535 which is the moiety including the receptor binding domain is included. That is, gene encoding a protein in which the amino acid sequence from position 378 to position 535 of diphtheria toxin had been fused to GST (Gluthathione-S-transferase) was made, and this was expressed in Escherichia coli to make a fusion protein (GST-DT) having the above structure. GST-DT inhibited the binding of ¹²⁵I-labeled diphtheria toxin to HB-EGF in a dose dependent manner. It has been found from the levels of the inhibition that GST-DT binds to HB-EGF with similar strength to diphtheria toxin. Therefore, it has been found that the amino acid sequence from position 378 to position 535, i.e., the moiety including the receptor binding domain is necessary and enough to the binding.

Here, it can be measured by the inhibition of binding of ¹²⁵I-labeled diphtheria toxin to HB-EGF as the above whether the protein has the activity to inhibit the binding of HB-EGF to the EGF receptor.

Thus, the protein having the activity to inhibit the binding of HB-EGF to the EGF receptor and substantially not having the toxicity of diphtheria toxin can be obtained by making a diphtheria toxin mutant protein retaining the receptor binding domain and having the mutated catalytic domain or a protein retaining the receptor binding domain of diphtheria toxin and deleting a part or all of the catalytic domain.

Examples of such a mutant include CRM197, DT52E148K and GST-DT. These do not have the toxicity of diphtheria toxin substantially and inhibit the binding of HB-EGF to the EGF receptor. CRM197 is the mutant obtained by mutating Gly at position 52 to Glu when counted by excluding the signal sequence of 25 amino acids. DT52E148K is the mutant obtained by mutating Glu at position 148 to Lys in addition to the above mutation when counted by excluding the signal sequence. GST-DT is the protein obtained by fusing the amino acid sequence from position 378 to position 535 when counted by excluding the signal sequence of diphtheria toxin to GST. The amino acid seuence for CRM197 (first 25 sequence represents the signal sequence) and the base sequence of the gene encoding it are shown in SEQ ID NO:3 and SEQ ID NO:4, respectively. The amino acid sequence of GST-DT (SEQ ID NO:5) and the base sequence (SEQ ID NO:6) of the gene encoding it are shown in FIGS. 5 and 6, respectively.

Here, it has been already reported that CRM197 does not have the toxicity of diphtheria toxin, i.e., does not have the ADP-ribosylation activity (T. Uchida and A. M. Pappenheimer Jr. (1972) Science 175, 901-903). It has been known that the 148K mutant having the mutation at 148E has the quite faint activity (J. T. Barbieri and R. J. Collier (1987) Infect. Immun. 55, 1647-1651). DT52E148K is a double mutant obtained by further adding the mutation of 148K to CRM197 which is the 52E mutant, and is preferable as the safer mutant. It has been confirmed that the toxicity of these mutants has no significant difference from the value of the sample in which diphtheria toxin was not added in the above protein synthesis inhibition experiment. But, as described above, it has been demonstrated by the detailed analysis by the present inventors that even these mutants have the remaining extremely faint toxicity of diphtheria toxin.

It is obvious that GST-DT has no toxicity of diphtheria toxin because it completely lacks the catalytic action domain.

A fragment containing the receptor binding domain can be made by synthesizing a DNA encoding the receptor binding domain by PCR method using the gene (P□197) encoding CRM197 as a template, inserting this into a multiple cloning site of an expression vector (pGEX-3X, pQE-30) for synthesizing a GST fusion protein or a histidine tag, incorporating the obtained plasmid in Escherichia coli and synthesizing a plasmid-encoded gene in Escherichia coli. The mutant having a mutation in the catalytic domain can be made as follows. A CRM197 region is synthesized by PCR using the gene encoding CRM197 incorporated in the plasmid (P□197) as the template and using a portion to be mutated as a primer. The primer is synthesized by introducing a point mutation so as to have the mutation, and used. The synthesized DNA is incorporated in a expression vector (pET-22b) for Escherichia coli to transform Escherichia coli, and the mutant is expressed in Escherichia coli.

The anti-cancer agent of the present invention can be used for the treatment of the entire malignant tumors such as breast cancer, prostate cancer, thyroid cancer and ovarian cancer, preferably can be used for the malignant tumors which express HB-EGF, and in particular can be used suitably for ovarian cancer, breast cancer and prostate cancer. The malignant tumor which has expressed HB-EGF can be identified by biopsy. In the case of ovarian cancer, the level of HB-EGF expression can be identified by collecting ascites.

The effect on breast cancer or prostate cancer can be demonstrated by examining the growth inhibitory effect on a breast cancer cell line such as MDA MB231 or a prostate cancer cell line such as PC-3.

The anti-cancer agent of the present invention includes a monoclonal antibody which has an activity to neutralize the toxicity of diphtheria toxin remaining in the above mutants of diphtheria toxin, and further comprises the monoclonal antibody against the above mutants of diphtheria toxin, which does not inhibit the binding of the above mutants of diphtheria toxin to the cells, and preferably, the monoclonal antibody against the above mutants of diphtheria toxin and the mutant of diphtheria toxin form the complex.

The above the diphtheria toxin mutant attenuates the toxicity of diphtheria toxin by the mutation and substantially does not have the toxicity of diphtheria toxin, but includes the case in which the toxicity of diphtheria toxin remains at extremely strict level as described above. Thus, the diphttheria toxin mutant can further lower the toxicity of diphtheria toxin by having the activity to neutralize the toxicity of diphtheria toxin and forming a complex with the monoclonal antibody which does not inhibit the binding of the above diphtheria toxin mutant to the cells.

The anti-diphtheria toxin mnoclonal antibody which has an activity to neutralize the toxicity of diphtheria toxin which remains in the diphtheria toxin mutant and does not inhibit the binding of the diphtheria toxin mutant to the cells (i.e., not inhibit the binding activity to proHB-EGF) can be made according to the standard method shown in the following reference (Hayakawa S, J. Biol. Chem. 258, 4311-4317, 1983). That is, such a monoclonal antibody can be made by separating a clone which produces the antibody which finally has the activity to neutralize the toxicity of diphtheria toxin but does not inhibit the binding of the diphtheria toxin mutant to the cells among clones which produce the antibody against the diphtheria toxin mutant. It is also possible to select one having the above natures for the diphtheria toxin mutant used in publicly known diphtheria toxin monoclonal antibodies.

The activity to neutralize the toxicity of diphtheria toxin can be easily measured by adding the complex of a diphtheria toxin mutant with the monoclonal antibody against the diphtheria toxin mutant to Vero-H cells and examining whether inhibition of colony formation is inhibited or not when compared with the diphtheria toxin mutant alone is added.

Meanwhile, it can be examined that the monoclonal antibody does not inhibit the binding of the diphtheria toxin mutant to the cells, by measuring the inhibition of the binding of ¹²⁵I-labeled diphtheria toxin to HB-EGF or measuring growth activity inhibition of HB-EGF in DER cells which are obtained by expressing an EGF receptor gene in IL-3-dependent 32D cells (obtained from ATCC) described later.

The monoclonal antibody against the diphtheria toxin mutant, which has the activity to neutralize the toxicity of diphtheria toxin which remains in the diphtheria toxin mutant and does not inhibit the binding of diphtheria toxin to the cells includes, for example, #2 ant-DT mAb (monoclonal antibody produced by a hybridoma of Deposit Number FERM P-19551 at International Patient Organism Depositary). The complex with the diphtheria toxin mutant can be made by mixing the diphtheria toxin mutant with the monoclonal antibody at an appropriate ratio.

The anti-cancer agent of the present invention can be formulated by directly using the above active components or combining with a pharmaceutically acceptable carrier for medical use.

The above anti-cancer agent can be administered orally or parenterally (e.g., intravenous, intramuscular, intraperitoneal, subcutaneous or intracutaneous injection, intrarectal administration, pemucosal administration, perairway administration). When applied to the malignant tumor such as ovarian cancer which intraperitoneally disseminates, it is preferable to administer by intraperitoneal injection in terms of being directly transported to the cancer cells.

Pharmaceutical compositions suitable for the oral administration include, for example, tablets, granules, capsules, powders, solutions, suspensions and syrups, and the pharmaceutical compositions suitable for the parenteral administration include, for example, injection agents, drops, suppositories and percutaneous absorbers, but the formulations are not limited thereto.

Types of additives for the formulation used for producing the above anti-cancer agent are not particularly limited, and those skilled in the art can optionally select them. For example, it is possible to use excipients, disintegrants or disintegrant aids, binders, lubricants, coating agents, bases, dissolving agents or dissolving aids, dispersants, suspending agents, emulsifiers, buffers, anti-oxidants, preservatives, isotonic agents, pH adjusters, stabilizers and the like, and specific components used for these purposes are well-known by those skilled in the art.

The additives for the formulation used for preparing the formulation for the oral administration include excipients such as glucose, lactose, D-mannitol, starch or crystalline cellulose; disintegrants or disintegrant aids such as carboxymethylcellulose, starch and calcium carboxymethylcellulose; binders such as hydroxypropylcellulose, hydroxymethylcellulose, polyvinyl pyrrolidone or gelatin; lubricants such as magnesium stearate or talc; coating agents such as hydroxypropylmethylcellulose, saccharose, polyethylene glycol, or titanium oxide; and bases such as petrolatum, liquid paraffin, polyethylene glycol, gelatin, kaolin, glycerine, purified water or hard fat.

The additives for the formulation usable for preparing the formulation for the injection or drop infusion include aqueous dissolving agents or dissolving agent aids or those capable of configuring dissolved injection agents in use such as distilled water for the injection, saline, and propylene glycol; isotonic agents such as glucose, sodium chloride, D-mannitol and glycerine; and pH adjusters such as inorganic acids, organic acids, inorganic bases or organic bases.

The amount of the active component contained in the above anti-cancer agent of the present invention is different depending on a dosage and an administration route, and can not be categorically defined, but can be determined typically by optionally selecting from the range of about 0.0001 to 70% in the final formulation.

The anti-cancer agent of the present invention can be administered to mammalian animals including human.

The amount of the anti-cancer agent of the invention to be administered should be optionally increased or decrease depending on conditions such as age, gender, body weight, symptom of a patient and administration route, and as the amount of the active component per adult per day, it is preferable to be in the range of about 1 μg to 30 mg per kg of the body weight. The pharmaceutical composition at the above dosage may be administered once daily or by dividing into several times, or may be administered once several days or several weeks sporadically, or may be administered with the component such as steroid to suppress the side effect.

### BEST MODES FOR CARRYING OUT THE INVENTION

Examples of the present invention will be described below, but the invention is not limited to these Examples.

### EXAMPLE 1

### Production of CRM197 Protein

Diphtheria bacillus C7(beta197)M1 obtained by lysogenizing a lysogenic bacterium stock (C7(□197) phage of C7(β197) (available from ATCC [American Type Culture Collection] Bacteria collection(No.39255) is cultured, and a bacterium solution at a logarithmic growth late phase is added to C-Y medium to which filtrated 2% maltose has been added so that an initial OD590 is 0.05. This OD corresponds to about 5×10⁷ microbial cells/ml. A flask is placed on a rotary shaker at 200 rotations per minutes, and cultured at 35° C. for 16 to 17 hours. When the OD reaches 10 to 15, the culture is terminated.

The above C-Y medium is prepared as follows. That is, 10 g of casamino acid, 20 g of yeast extract solution and 5 g of KH₂PO₄ are dissolved in 1 liter of distilled water, then 2 ml of 50% CaCl₂.2H₂O is added and subsequently pH is adjusted to 7.4. The solution is boiled and then filtrated. Subsequently, 2 ml of Mueller and Miller's solution II (22.5 g of MgSO₄, 0.115 g of β-alanine, 0.115 g of nicotinic acid, 7.5 mg of pimelic acid, 1 g of CuSO₄, 1 g of ZnSO₄.5H₂O, 1 g of MnCl₂/100 ml of H₂O) and 1 ml of Mueller and Miller's solution III (20 g of L-cystine and 20 ml of concentrated hydrochloric acid/100 ml of H₂O) are added thereto. An aliquot of 100 ml was dispensed, and autoclaved to obtain the C-Y medium.

CRM197 protein is purified as follows.

The culture medium is centrifuged at 10,000 g for 15 minutes. Ammonium sulfate is added at a final concentration of 65% saturation to a culture supernatant. The mixture is left stand in a cold room for 24 to 48 hours. A precipitate is collected, dissolved in 0.02 M Tris hydrochloride buffer (pH 7.2), and dialyzed against the same buffer.

The solution is centrifuged to remove insoluble materials, then applied on DE52 column, and then eluted with concentration gradient of NaCl in 0.02 M Tris hydrochloride buffer (pH 7.2). CRM197 is eluted at 0.08 M of NaCl. Ammonium sulfate is added to make an eluted solution at 65% saturation. A precipitate is dissolved in 0.01 M Tris hydrochloride buffer and equilibrated again. The solution is applied on the DE52 column again, and an eluate is precipitated with ammonium sulfate again. Subsequently the solution is applied on Sephacryl S-200 column, and eluted with a solution of HEPES-NaOH, pH 7.2 and 0.15 M NaCl. Eluted CRM197 is applied on DeToxi gel to remove an LPS-like substance contained in the CRM197 sample, and then used for experiments. In absorbance of CRM197 at 280 nm, 1 OD corresponds to about 0.67 mg/ml.

### EXAMPLE 2

### Tumorigenicity Experiments were Performed using Nude Mice

An ovarian cancer cell line, SKOV-3 (available from ATCC) cultured in RPMI1640+10% FBS was washed with PBS(−) and collected using 0.25% trypsin. The cells were washed twice with RPMI1640+10% FBS and twice with serum free RPMI1640, 5×10⁶ cells were suspended in 250 μl of RPMI1640 (with serum), and this was inoculated by subcutaneously injecting in back of the nude mouse.

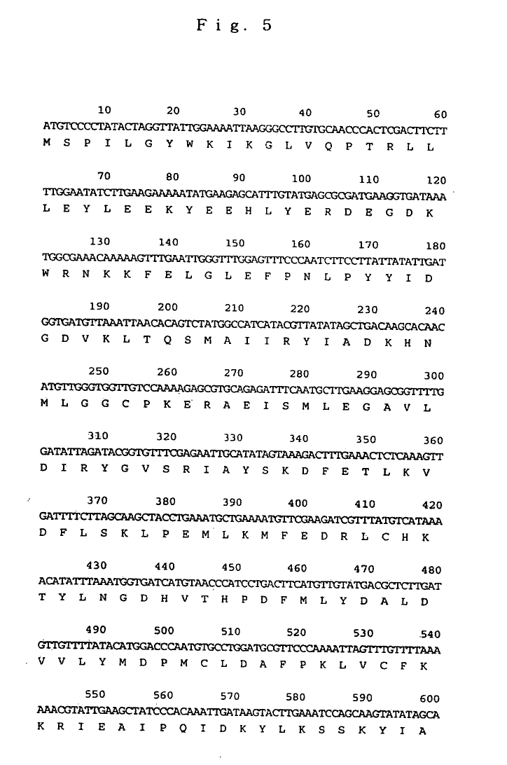

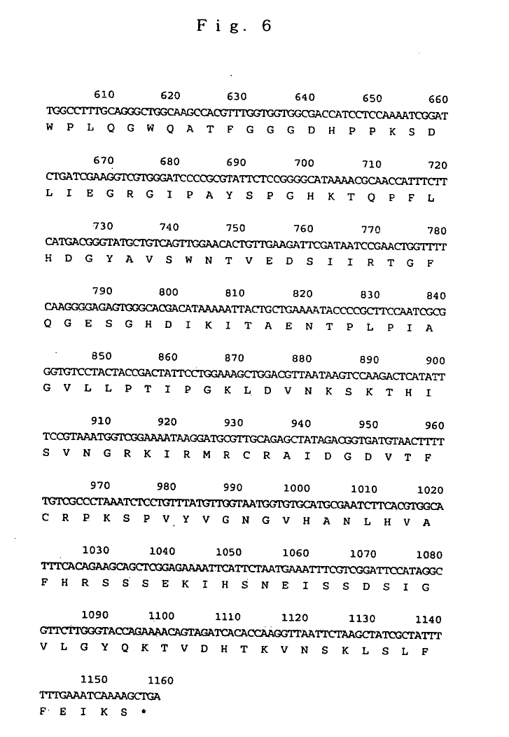

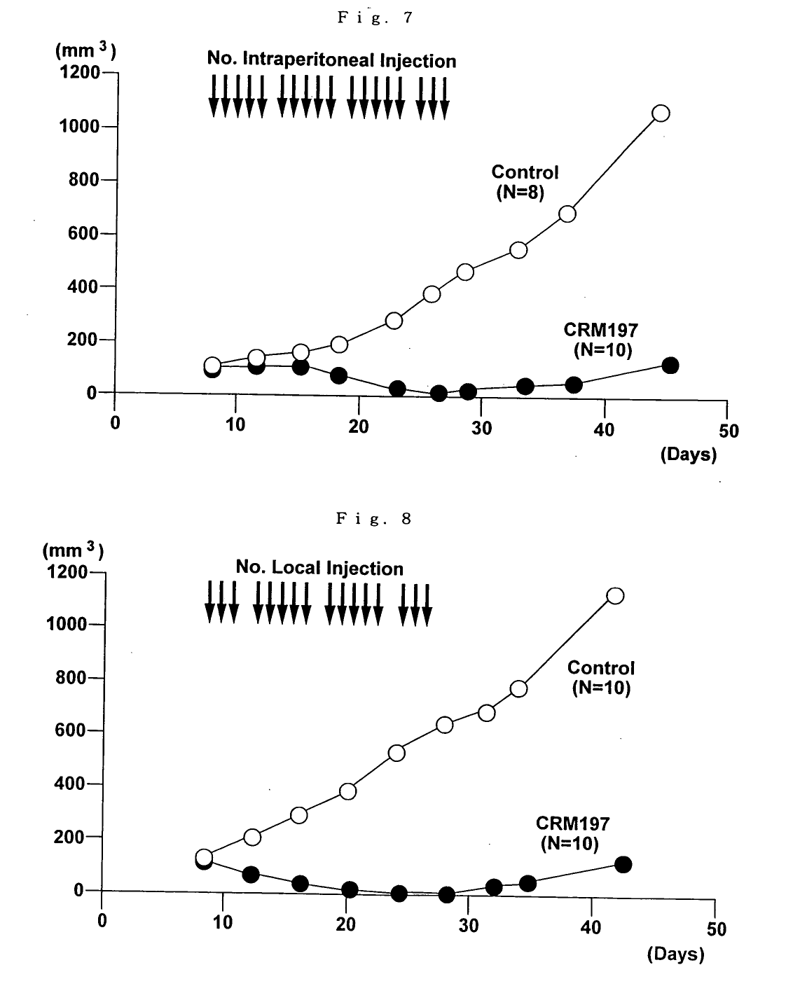

For the nude mice in one group, the administration of CRM197 was started from one week after inoculating SKOV-3 cells, and CRM197 was administered intraperitoneally at 200 μg/day five times a week over 4 weeks (FIG. 7). For the nude mice in another group, the administration of CRM197 was started from one week after inoculating the SKOV-3 cells, and CRM197 was administered directly into the inoculated tumor at 100 μg/day five times a week over 4 weeks (FIG. 8). In addition, for the nude mice in another group, the administration of CRM197 was started from 20 days after inoculating the SKOV-3 cells, and CRM197 was administered directly into the inoculated tumor at 100 μg/day five times a week over 3 weeks (FIG. 9). In all cases, the nude mice to which CRM197 had not been administered were controls. Relationships between the administered time period and the tumor volume are shown in FIGS. 7 to 9. A major axis and a minor axis of the growing tumor were measured every 3 to 4 days, and the tumor volume was calculated by the major axis x the minor axis x the minor axis x ½.

From these results, it was found that the growth of the tumor was inhibited by the administration of CRM197 in all cases.

### EXAMPLE 3

The ovarian cancer cell line RMG-1 cell (registered in Japanese Collection of Research Bioresources Cell Bank), the ovarian cancer cell line OV47 cell, and SKOV-H cells made by transfecting the ovarian cancer cell line SKOV-3 cells with the HG-EGF gene were collected as was the case with the SKOV-3 cells in Example 2, and inoculated in the back of nude mice by subcutaneous injection in place of the SKOV-3 cells.

In all cases, 1 mg of CRM197 was intraperitoneally injected immediately after and one week after the inoculation. The nude mice to which CRM197 had not been administered were the controls.

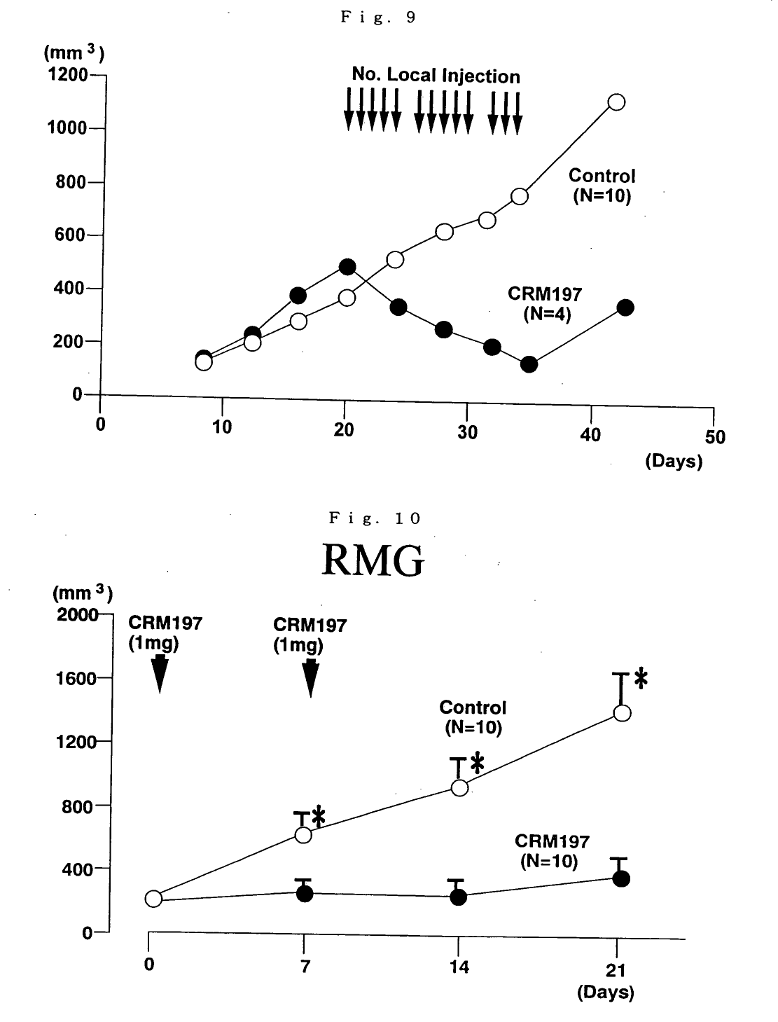

The relationships between the administered time period and the tumor volume are shown in FIGS. 10 to 12. The cases of the nude mice to which the RMG-1 cells, OV47 cells and SKOV-H cells were inoculated are shown in FIG. 10, FIG. 11 and FIG. 12, respectively.

From these results, it was found that the growth of the tumor was inhibited by the administration of CRM197 in all cases.

### EXAMPLE 4

### Cytotoxicity Test of CRM197

In the conventional experiments, ADP-ribose transferase activity was not observed at all in the fragment A of CRM197, and it has been believed that CRM197 is a non-toxic molecule. However, it is important in the present invention to elucidate whether CRM197 has no toxicity or has only faint toxicity as diphtheria toxin. Thus, the ADP-ribose transferase activity in CRM197 was analyzed in more detail.

First, in order to examine the cytoxicity of CRM197, CRM197 at various concentrations was added to Vero cells (available from ATCC) and Vero-H cells (human HB-EGF cDNA supplied from OriGene Technologies was cloned into pCDNA3.1 plasmid supplied from Invitrogen, which was then transfected) obtained by expressing HB-EGF at high level in Vero cells, and the cells were cultured for a week to examine colony formation rates.

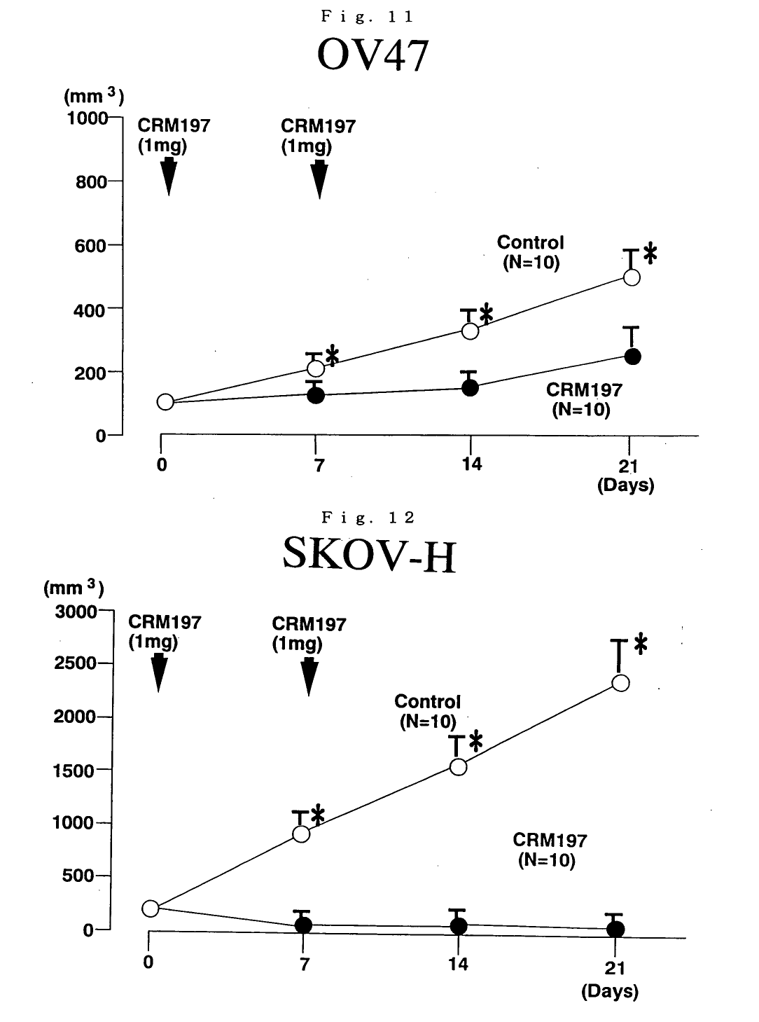

FIG. 13 shows the colony formation rates obtained by the following procedures. Vero cells and Vero-H cells were seeded in 6well plates at a density of 300 cells/well, cultured for 10 hours, CRM197 was added thereto, the cells were cultured for 24 hours, subsequently cultured in CRM197-free medium for 6 days, and then the numbers of the colonies were counted. FIG. 14 shows the colony formation rates when CRM197 was added to Vero cells and Vero-H cells, and the cells were cultured for one week. As a result, in Vero-H cells, the decrease of the colony formation rate was observed at 1 μg/ml of CRM197 when cultured with CRM197 for 24 hours and at 100 ng/ml of CRM197 when cultured in the presence of CRM197 for one week. No significant decrease of the colony number was observed in Vero cells under both conditions. CRM197 exhibited stronger toxicity for Vero-H cells than Vero cells. Thus, this toxicity was suggested to be involved in the diphtheria toxin receptor (proHB-EGF). When diphtheria toxin in place of CRM197 was allowed to act upon the cells for 24 hours and subsequently the cells were cultured in diphtheria toxin-free medium for 6 days, the colony scarcely appeared at a concentration of about 1 fg/ml (about 3% of the colony number obtained when no toxin was added). Therefore, the cytotoxicity of CRM197 measured by the colony formation method used here is 1/10¹⁰ or less of the toxicity of diphtheria toxin, and it was found that the toxicity of CRM197 was extremely faint.

### EXAMPLE 5

### Protein Synthesis Inhibition Experiment by CRM197

The toxic action of diphtheria toxin is a protein synthesis inhibition based on ADP-ribosylation of EF-2. Thus, in order to investigate whether the toxicity of CRM197 is owing to the protein synthesis inhibition or not, the inhibitory activity of protein synthesis by CRM197 was examined. CRM197 was exposed to Vero cells and Vero-H cells used in Example 4, and its inhibitory activity of the protein synthesis was examined by uptake of [³H]Leu in the protein. Specifically, Vero cells and Vero-H cells were seeded in 24-well plates at a density of 1×10⁵ cells/ml, cultured for 16 hours, then CRM197 was added thereto, and the cells were further cultured for 36 hours. Subsequently, [³H]Leu was added, the cells were incubated for one hour, and the radioactivity of [³H]Leu incorporated into the protein was measured using a liquid scintillation counter to obtain the inhibitory activity of the protein synthesis.

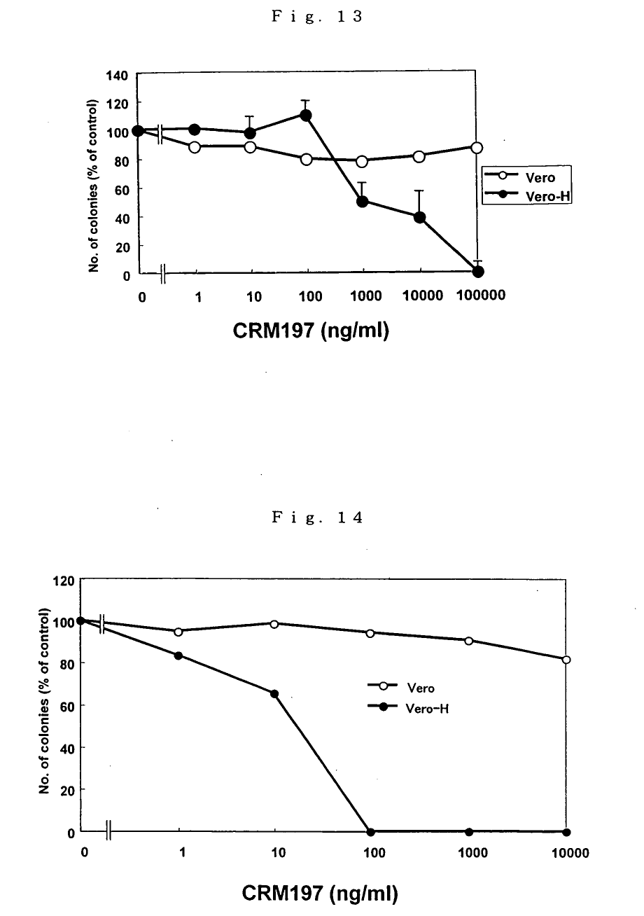

As a result, in the Vero-H cells, the inhibition of the protein synthesis was observed at concentrations of 100 ng/ml or more of CRM197. In the Vero cells, the inhibition of the protein synthesis was scarcely observed under this condition (FIG. 15).

### EXAMPLE 6

### Cytotoxicity Test of DT52E148K

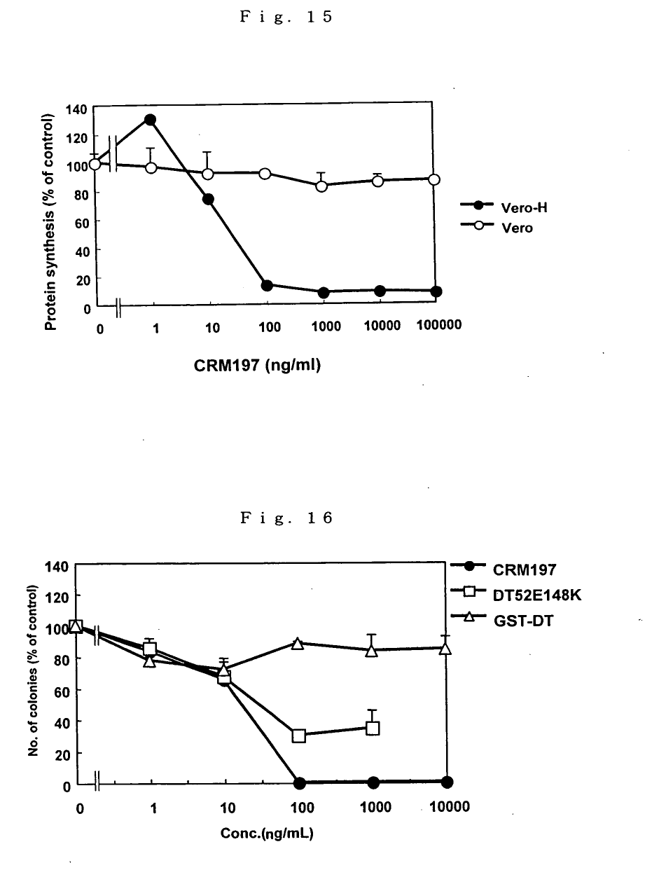

Concerning DT52E148K which is the mutant having two mutations in diphtheria toxin and the recombinant protein GST-DT, their toxicity was measured by the colony formation method as with Example 4. As a result, DT52E148K exhibited lower toxicity for the Vero-H cells than CRM197 (FIG. 16). Meanwhile, no cytotoxicity was observed in the Vero cells (FIG. 17). Therefore, to completely eliminate the toxicity, only two mutations were shown to be insufficient. GST-DT exhibited no toxicity for both the Vero cells and the Vero-H cells.

### EXAMPLE 7

### Resistance of Vdtr Cells to CRM197 and DT52E148K

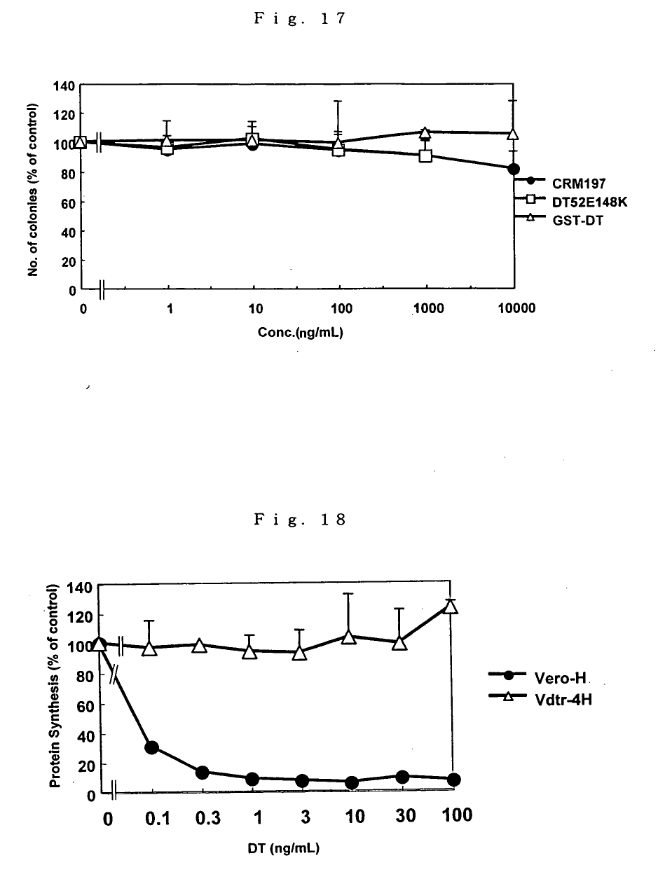

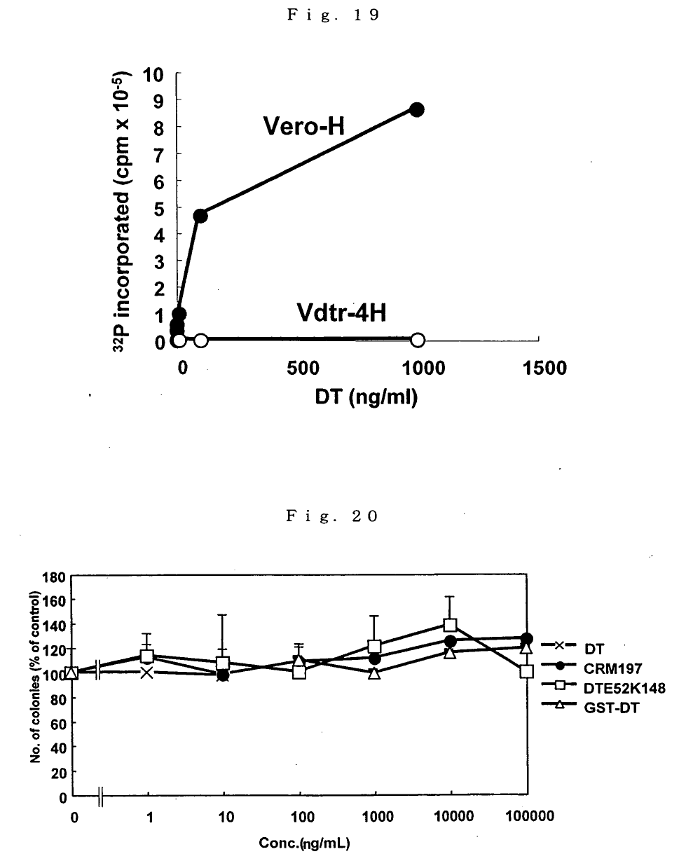

In order to investigate whether the cytotoxicity and the protein synthesis inhibition shown until now of CRM197 and DT52E148K are attributed to inactivation of EF-2 owing to ADP ribosylation activity which diphtheria toxin has, a diphtheria toxin resistant cell line, Vdtr cell was made. Specifically, according to the following references (Moehring J M and Moehring T J, Somat. Cell Genet. 5, 453-468, 1979: Kohno K et al, Somat. Cell Genet. 11, 421-423, 1985), the Vero cells were treated with EMS, subsequently cultured with diphtheria toxin and surviving cells were obtained. Among them, cells which were resistant to high concentrations of diphtheria toxin were selected to obtain Vdtr cell. Subsequently, HB-EGF was expressed at high level in the Vdtr cells to obtain Vdtr-4H cells (human HB-EGF gene supplied from OriGene Technologies was cloned into pCDNA3.1 plasmid supplied from Invitrogen, which was then transfected). The inhibition rate of the protein synthesis when diphtheria toxin was added to the Vdtr-4H cells is shown in FIG. 18, and the result of ADP-ribosylation assay in a cell free system obtained by adding the fragment A to cell lysate of this cell lines is shown in FIG. 19. In this cell, ADP-ribosylation of EF-2 was not observed at all in the ADP-ribosylation assay in the cell-free system. Thus, it was shown that diphtheria toxin resistance in this cell was attributed to EF-2.

In order to identify whether the cytotoxicity of CRM197 is owing to the ADP-ribosylation activity, the toxicity of CRM197 to the Vdtr-4H cells was investigated. CRM197 was added to the Vdtr-4H cells, and its toxicity was investigated by the same colony formation method as in Example 4. As a result, even 100 μg/ml of CRM197 did not exhibit the toxicity for the Vdtr-4H cells at all (FIG. 20). Likewise, DT52E148K did not exhibit the toxicity at all (FIG. 20). From these results, it was revealed that the cytotoxicity and the protein synthesis inhibition which CRM197 exhibited was owing to ADP ribosyl transferase in the fragment A.

### EXAMPLE 8

### ADP-Ribosylation Experiments of EF-2 in Cell Free System

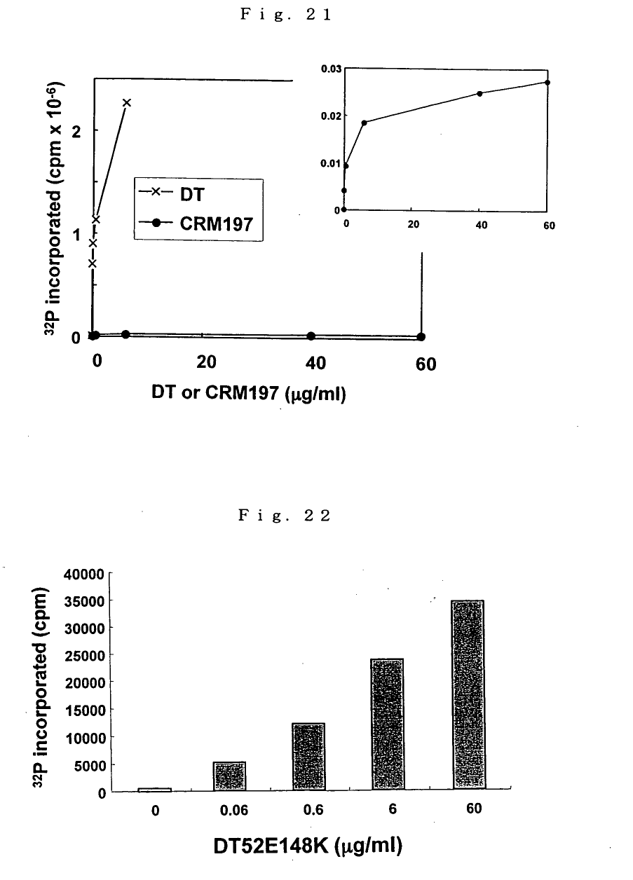

Experimental results using the Vdtr cells in Example 7 indicate that the cytotoxicity present in CRM197 and DT52E148K is owing to the residual ADP-ribosyl transferase activity. In order to further confirm this, ADP-ribosylation experiments were performed under the cell-free condition. Using the method shown in the following reference (Gill, D M and Pappenheimer, A M Jr. J. Biol. Chem. 246, 1492-1495, 1971), CRM197 or Dt52E148K was added to EF-2 extracted from rabbit liver, [³²P]NAD was added thereto, and the mixture was incubated at 37° C. for 10 minutes to perform the ADP-ribosylation reaction in the cell free system. Subsequently, the radioactivity was measured using a liquid scintillation counter. As a result, although the activity was quite faint, the ADP-ribosylation activity of EF-2 was observed in CRM197 and DT52E148K (FIGS. 21 and 22). In a right upper figure in FIG. 21, the ADP-ribosylation activity in CRM197 was shown by magnifying a scale of a vertical axis. From these results, it was concluded that the activity of ADP-ribosylating EF-2 remained only slightly in both CRM197 and DT52E148K.

### EXAMPLE 9

### Neutralization of the Cytotoxicity of CRM197 by Anti-DT mAb #2

When CRM197 is used as an inhibitor of HB-EGF growth activity, it is not preferable in some cases that CRM197 has the cytotoxicity although it is faint. Thus, next, the condition to inhibit the remaining toxicity in CRM197 was investigated. Many monoclonal antibodies against diphtheria toxin have been isolated (Hayakawa S, J. Biol. Chem. 258, 4311-4317, 1983). Among them, there are antibodies which inhibit the cytotoxicity of diphtheria toxin but do not inhibit the binding of diphtheria toxin to the receptor. Among them, it was found that #2 anti-DT mAb neutralized the toxicity of CRM197 as was the case with diphtheria toxin but did not inhibit the binding of CRM197 to HB-EGF.

#2 Anti-DT mAb (monoclonal antibody produced by the hybridoma of Deposit Number FERM P-19551 at International Patent Organism Depositary) was added simultaneously with CRM197 to investigate whether the toxicity of CRM197 was neutralized or not.

The production of the monoclonal antibody against diphtheria toxin is shown by the following reference (Hayakawa S, J. Biol. Chem. 258, 4311-4317, 1983), and briefly is as follows. Diphtheria toxin (0.1 mg) treated with formalin was inoculated with Freunds adjuvant into peritoneal of a BALB/c mouse, and this was performed total three times every one week. Several days after the final inoculation, spleen cells were removed from this mouse, and fused with murine myeloma cells SP2/0. The fused cells were cultured in HAT selection medium, and clones which produced the antibody against diphtheria toxin, the clone which produced the antibody which had the activity to neutralize the toxicity of diphtheria toxin but did not inhibit the binding of diphtheria toxin to the cells was finally isolated.

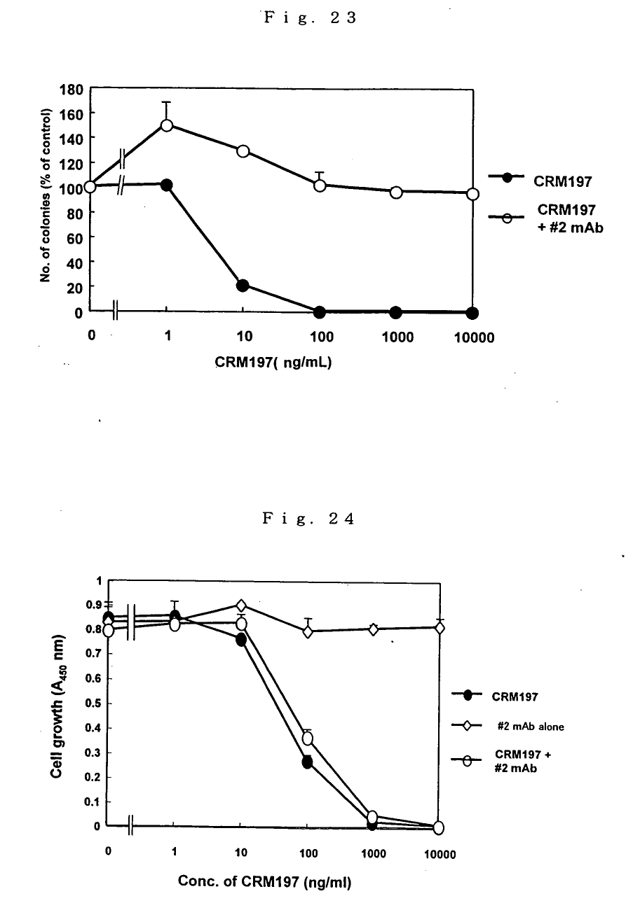

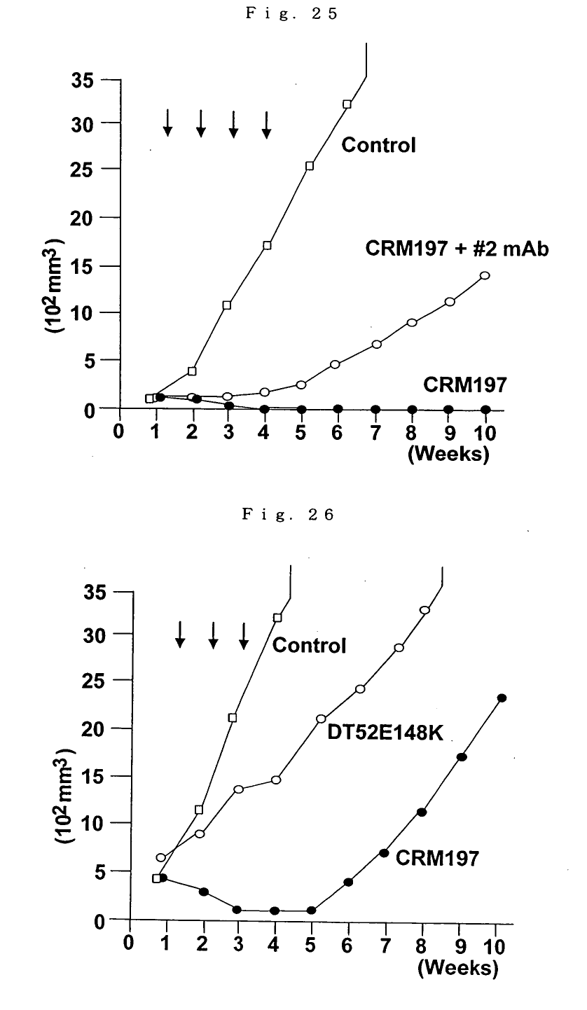

The complex of CRM197/#2 anti-DT mAb was made by first mixing CRM197 (1 mg) and #2 anti-DT mAb (10 mg), and then incubating the mixture at 37° C. for one hour. This complex at various concentrations was added to the Vero-H cells, which were then cultured for one week to examine the colony formation rate. As a result, when CRM197 alone was used, the cells died out at 100 ng/ml of CRM197, but when the complex of CRM197/#2 anti-DT mAb was used, the colony formation was not inhibited at a maximum dose of 10 μg/ml. Thus, it was found that #2 anti-DT mAb completely inhibited the cytotoxicity of CRM197 (FIG. 23).

### EXAMPLE 10

### A Complex of CRM197/#2 Anti-DT mAb Possesses Inhibitory Activity to HB-EGF Growth Activity

The action to inhibit the growth activity of HB-EGF of the complex of CRM197/#2 anti-DT mAb was investigated. EGFR gene (supplied from OriGene Technologies) was cloned into pCDNA3.1 plasmid (supplied from Invitrogen). DER cell was made by transfecting EGFR gene in 32D cell available from ATCC) which exhibited a growth ability depending on IL-3. DER cell can grow by the growth activity of HB-EGF even in the absence of IL-3. CRM197 or the complex of CRM197/#2 anti-DT mAb was added to this cell in the presence of HB-EGF. The cells were cultured for 48 hours, and the number of the grown cells was measured by MTT assay. The DER cells grew in the absence of CRM197 or the complex of CRM197/#2 anti-DT mAb, but the growth of the DER cells was inhibited in the presence of CRM197 or the complex of CRM197/#2 anti-DT mAb were compared, there was almost no difference between the both, and they showed the similar inhibitory effects. That is, it was found that the complex of CRM197/#2 anti-DT mAb inhibited the cytotoxicity of CRM197 but retained the same inhibitory activity as in CRM197 alone on the growth activity of HB-EGF.

### EXAMPLE 11

### Tumor Suppression Effect by Complex of CRM197/#2 Anti-DT mAb

For nude mice (3 mice) in one group, the SKOV-H cells were inoculated, one week after the inoculation, the administration of CRM197 (1 mg/mouse/week) or the complex of CRM197/#2 anti-DT mAb (including 1 mg of CRM197/week) was started, and it was administered intraperitoneally once a week over 4 weeks. The complex of CRM197/#2 anti-DT mAb was prepared by incubating 1 mg of CRM197 and 8 mg of #2 anti-DT mAb at room temperature for one hour to use. The nude mice to which CRM197 had not been administered were the controls. The relationship between the administration of CRM197 or the complex of CRM197/#2 anti-DT mAb and the tumor volume is shown in FIG. 25. The tumor volume was measured by the same way as in Example 2. From this experiment, it was revealed that the complex of CRM197/#2 anti-DT mAb inhibited the growth of the tumor, but it was shown that its effect was weaker than CRM197 alone.

The complex of CRM197/#2 anti-DT mAb completely inhibits the faint cytotoxicity of CRM197 as shown in figures, and thus, can be anticipated to be safer than CRM197 alone. Meanwhile, when CRM197 alone is used, the effect to inhibit the growth of the tumor is stronger. It appears that this is because the faint cytotoxicity of CRM197 was added in addition to the HB-EGF growth activity inhibition of CRM197. Therefore, when CRM197 is used, if the safety is more important, it is possible to administer as the complex with #2 anti-DT mAb, and if the effect is more important, the administration of CRM197 alone is also effective.

### EXAMPLE 12

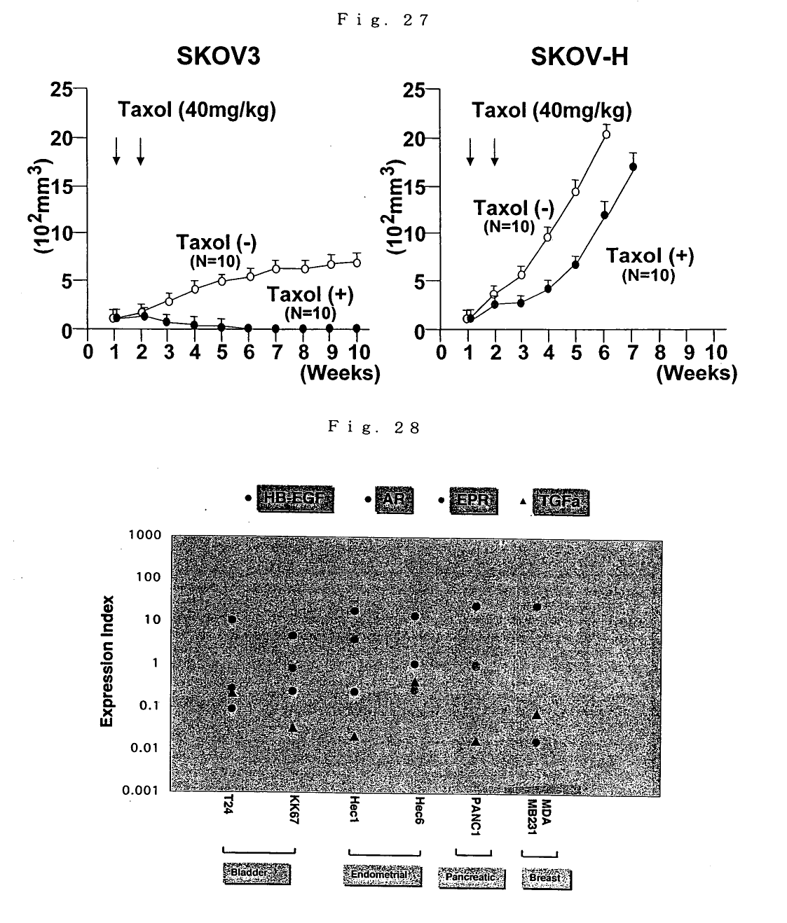

The inhibitory effect on the tumor was examined by the same way as in Example 11, except that DT52E148K or CRM197 was administered once a week over 3 weeks. The relationship between the administration of DT52E148K or CRM197 and the tumor volume is shown in FIG. 26. It was revealed from this experiment that DT52E148K inhibited the growth of the tumor, but it was shown that its effect was weaker then CRM197. This result for DT52E148K which has two mutations in the fragment A having the ADP-ribose transferease activity and exhibits the low cytotoxicity confirms the speculation that CRM197 has the inhibitory effect on the tumor growth by the faint cytotoxicity of CRM197 in addition to the inhibitory effect of CRM197 on growth activity of HB-EGF.

### REFERENCE EXAMPLE

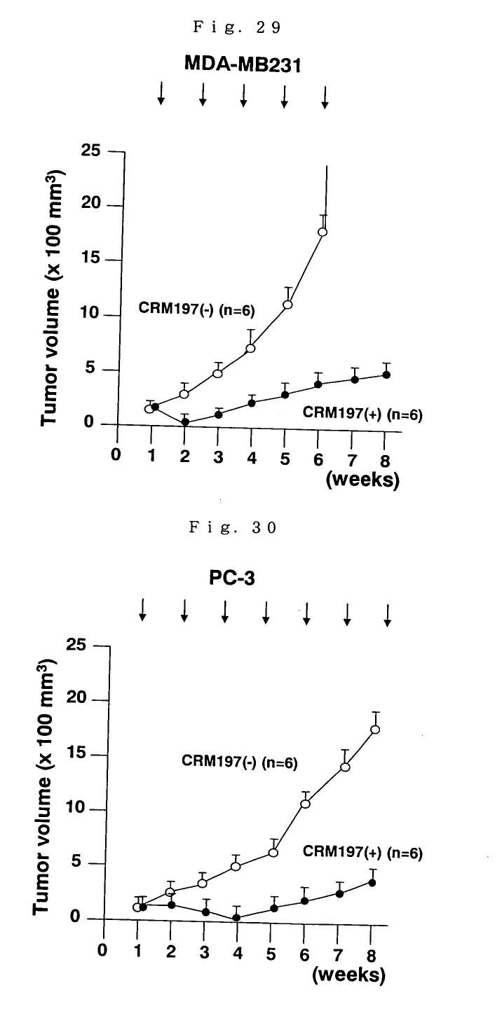

For the nude mice (10 mice) in one group, the SKOV-3 cells or the SKOV-H cells were inoculated, and one and two weeks after the inoculation, 40 mg/week of taxol was administered intraperitoneally. The results are shown in FIG. 27. It was found that taxol was effective for the SKOV-3 cells whereas less effective for the SKOV-H cells. It was found that taxol had an action mechanism different from the anti-cancer agent of the present invention because HB-EGF was abundantly expressed in the SKOV-H cells. Therefore, it is suggested that the anti-cancer agent of the present invention is effective for the cases for which taxol is not so effective.

### Measurement of Expression Levels of EGFR Ligand in Various Cancer Cells (FIG. 28)

RNA was extracted from cells of T24, KK67 (urinary bladder cancer), hec1, Hec6 (endometrial cancer), PANC1 (pancreatic cancer) and MDA MB231 (breast cancer), and expression amounts of HB-EGF, TGFα, amphiregulin and epiregulin were measured by a real-time PCR method. An expression index was displayed as a value obtained by dividing a copy number of the EGF ligand by a copy number of glyceraldehyde-3-phosphate dehydrogenase (GAPDH) and multiplying an obtained quotient by 10,000. The results are shown in FIG. 28.

As shown in FIG. 28, HB-EGF exhibited the highest value in all cancer cells, and its expression indices were around 10. AR exhibited the expression index which was the lowest in T24, was the next lowest to TGFα in KK67 and Hec1, and the second highest in Hec6. EPR and AR exhibited the similar expression index in PANC1. EPR exhibited the expression index which was the second highest in T24, KK67 and Hec1 and the lowest in Hec6 and MDA MB231.

From the results in FIG. 28, it was shown that HB-EGF was expressed at extremely high levels in the cancer cells such as MDA MB231 (breast cancer), and it was suggested that CRM197 was effective.

### EXAMPLE 13

### Tumorigenicity Experiments using Nude Mice were Performed

A human breast cancer cell line, MDA MB231 (available from Human Science Research Resources Bank) cultured in RPMI+10% FBS was washed with EDTA PBS(−), and collected using 0.25% trypsin. The cells were washed twice with RPM11640+10% FBS and twice with serum free RPMI1640, 5×10⁶ cells were added to 250 μl of RPMI1640 (, and this was inoculated in back of the nude mouse by subcutaneous injection. For the nude mice in one group, from 7 days after the cell inoculation, 50 mg/kg/week of CRM197 was intraperitoneally administered once a week over 8 weeks. The nude mice to which CRM197 had not been administered were the controls. The relationship between the administered time period and the tumor volume is shown in FIG. 29. Here, a major axis and a minor axis of the growing tumor were measured every one week, and the tumor volume was calculated by the major axis x the minor axis x the minor axis x ½.

### EXAMPLE 14

### Tumorigenicity Experiments using Nude Mice were Performed.

A human prostate cancer cell line, PC-3 (available from Human Science Research Resources Bank) cultured in RPMI1640+10% FBS was washed with EDTA PBS(−), and collected using 0.25% trypsin. The cells were washed twice with RPMI1640+10% FBS and twice with serum free RPMI1640, 5×10⁶ cells were added to 250 μl of RPMI, and this was inoculated in the back of the nude mouse by subcutaneous injection. For the nude mice in one group, from 7 days after the cell inoculation, 50 mg/kg/week of CRM197 was intraperitoneally administered once a week over 8 weeks. The nude mice to which CRM197 had not been administered were the controls. The relationship between the administered time period and the tumor volume is shown in FIG. 30. Here, a major axis and a minor axis of the growing tumor were measured every one week, and the tumor volume was calculated by the major axis x the minor axis x the minor axis x ½.

### EXAMPLE 15

### Peritoneal Dissemination Model

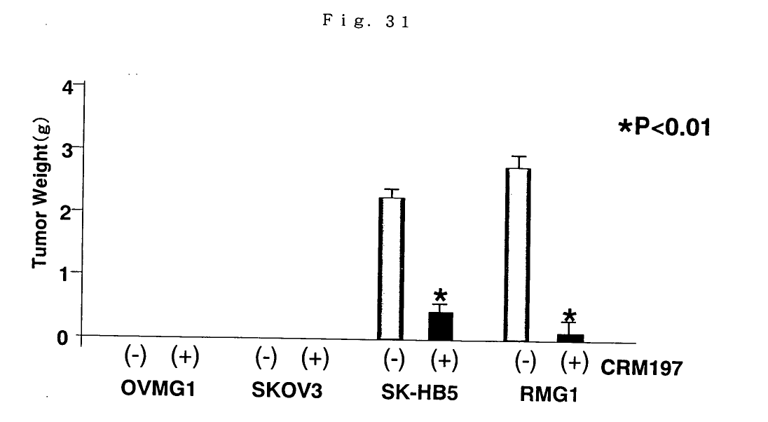

Cells at 1×10⁷ of a human ovarian cancer cell line SK-HB5 or RMG-1 were intraperitoneally inoculated in the nude mouse. For the nude mice in one group, from 7 days after the cell inoculation, 50 mg/kg/week of CRM197 was intraperitoneally administered total 5 times. In 6th week after the inoculation, abdominal operation was performed, and all intraperitoneal disseminated foci were removed to measure the total weight. The results are shown in FIG. 31.

The present invention can be utilized for producing the anti-cancer agent effective for the treatment of various cancers including breast cancer, prostate cancer, thyroid cancer and ovarian cancer.

1. A anti-cancer agent comprising as an active component a diphtheria toxin mutant protein, wherein said protein inhibits binding of HB-EGF to an EGF receptor and substantially does not have toxicity of diphtheria toxin.

2. The anti-cancer agent according to claim 1, wherein the protein comprises at least the receptor binding domain in the amino acid sequence of diphtheria toxin without having a mutation.

3. The anti-cancer agent according to claim 1 wherein the protein comprises an amino acid sequence having one or more amino acid deletions, substitutions or additions in the amino acid sequence of diphtheria toxin.

4. The anti-cancer agent according to claim 1 wherein the protein is either CRM197 or DT52E148K.

5. The anti-cancer agent according to claim 1 having an activity to neutralize toxicity of diphtheria toxin which remains in said diphtheria toxin mutant and further comprising a monoclonal antibody against said diphtheria toxin mutant, which does not inhibit binding said diphtheria toxin mutant to cells, wherein the monoclonal antibody against said diphtheria toxin mutant and the diphtheria toxin mutant form a complex.

6. The anti-cancer agent according to claim 5 wherein the monoclonal antibody against said diphtheria toxin mutant is #2 anti-DT mAb.

7. The anti-cancer agent according to claim 1, wherein the cancer is ovarian cancer, breast cancer or prostate cancer.

8. The anti-cancer agent according to claim 1 wherein the cancer is peritoneal metastatic cancer.

9. A anti-cancer agent comprising as an active component a protein which is any of the following proteins (a), (b) or (c), inhibits binding of HB-EGF to an EGF receptor and substantially does not have toxicity of diphtheria toxin:
(a) a protein comprising part of diphtheria toxin and at least the receptor binding domain of diphtheria toxin;
(b) a protein comprising an amino acid sequence having one or more amino acid deletions, substitutions or additions in the amino acid sequence of the protein (a); and
(c) a complex protein comprising any of (a) and (b).

10. The anti-cancer agent according to claim 9 wherein any of the proteins (a), (b) and (c) does not have the catalytic domain of diphtheria toxin.

11. The anti-cancer agent according to claim 9 wherein the protein (c) is GST-DT.


SEQUENCE LISTING (4 sequences)

SEQ ID NO: 1 - protein, 560 aa, Diptheria
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - DNA, 1683 bp, Diptheria
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 3 - protein, 560 aa, Diptheria
MSRKLFASILIGALLGIGAPPSAHAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 4 - DNA, 1683 bp, Diptheria
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagagttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga